(19) United States
(12) Patent Application Publication
(10) Pub. No.: US 2012/0310106 A1
(43) Pub. Date: Dec. 6, 2012

(54) Device-independent neurological monitoring system

(76) Inventor: James Cavuoto, Truckee, CA (US)

(21) Appl. No.: 13/507,067

(22) Filed: Jun. 1, 2012

Related U.S. Application Data
(60) Provisional application No. 61/519,964, filed on Jun. 2, 2011.

(51) Int. Cl. A61B 5/0476

(52) U.S. Cl. 600/544

(57) ABSTRACT

A system that analyzes neurological signals is described. During operation, the system monitors neurological signals from users at multiple locations, where the neurological signals are associated with different electrode configurations. Then, the system modifies the monitored neurological signals to correct for the different electrode configurations so that a resulting set of modified neurological signals corresponds to a common electrode configuration, thereby facilitating subsequent identification of a subset of the set of modified neurological signals.

### CROSS REFERENCE TO RELATED APPLICATIONS

This application claims priority under 35 U.S.C. 119(e) to U.S. Provisional Application Ser. No. 61/519,964, entitled “Device-Independent Neurological Monitoring System,” filed on Jun. 2, 2011, by James Cavuoto, the contents of which are herein incorporated by reference.

### BACKGROUND

The present disclosure relates to techniques for a system for collecting neurological signals using different electrode or sensor configurations from users at multiple locations.

Neurological monitoring and stimulation is increasingly popular. For example, driven by the lower cost and increased availability of electrodes and sensors companies are exploring neuromarketing in which neurological data is collected from users while the users engage in activities, such as in a focus group. However, in order to collect the neurological data, these companies are often forced to bring the users to a common location, where a standard type of electrode or sensor model is used to monitor the users. This approach can be cumbersome and expensive. Consequently, it can restrict the number of users that participate, which may bias the collected neurological data.

### SUMMARY

The disclosed embodiments relate to a system (such as a computer system) that analyzes neurological signals. During operation, the system monitors neurological signals from users at multiple separate locations, where the neurological signals are associated with different electrode configurations. Then, the system modifies the monitored neurological signals to correct for the different electrode configurations so that a resulting set of modified neurological signals corresponds to a common electrode configuration so that a subset of the set of modified neurological signals can be identified.

Note that the different electrode configurations may include: different types of electrodes, such as different electrode models provided by different manufacturers; different spatial sampling rates; and/or different electrode positions on the users.

Moreover, the neurological signals may include electroencephalogram signals.

In some embodiments, the system analyzes the modified neurological signals based on physiological responses of the users to external stimuli to identify the subset of the set of modified neurological signals. For example, the external stimuli may be displayed audio-video information and the physiological responses may include: a behavior, an emotional response, a vital sign, a motion, an ability to perform a task, etc.

Another embodiment provides a method that includes at least some of the operations performed by a system.

Another embodiment provides a computer-program product for use with the system. This computer-program product includes instructions for at least some of the operations performed by the system.

### BRIEF DESCRIPTION OF THE FIGURES

FIG. 1 is system in accordance with an embodiment of the present disclosure.

FIG. 2 is a flow chart illustrating a method for analyzing neurological signals in accordance with an embodiment of the present disclosure.

FIG. 3 is a block diagram illustrating a computer system in the system of FIG. 1 in accordance with an embodiment of the present disclosure.

FIG. 4 is a block diagram illustrating a data structure for use in the computer system of FIG. 3 in accordance with an embodiment of the present disclosure.

Note that like reference numerals refer to corresponding parts throughout the drawings. Moreover, multiple instances of the same part are designated by a common prefix separated from an instance number by a dash.

### DETAILED DESCRIPTION

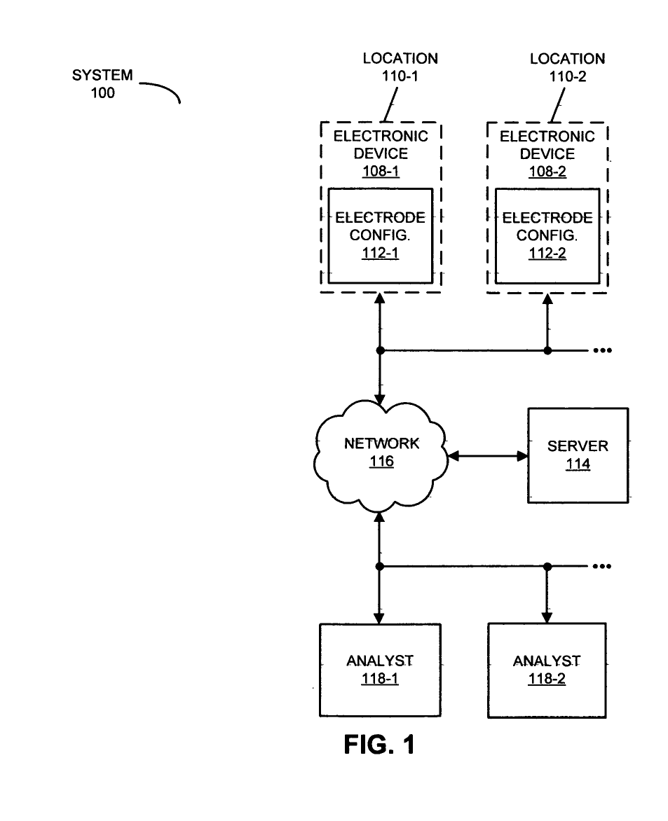

A system for multi-user interactive physiological monitoring/stimulation is described. As shown in FIG. 1, in this system neurological signals (or brainwave data) may be measured from or neurological stimulation may be applied to users at multiple separate locations 110 via electronic devices 108 (such as computers) and electrode configurations 112. For example, the measured neurological signals or the generated neurological stimulation may utilize: wireless electroencephalogram (EEG) headsets, brain-computer interfaces, etc. These measurements and/or stimulations may occur when the users interact with content, such as: text, pictures, video, music, sound, advertisements, sensory stimuli, video games, augmented reality, virtual reality, etc. In addition, the measurements may include: user feedback about the content, such as: approval, disapproval, descriptions, classifications, emotional responses, etc. In some embodiments, the measurements include user physiological data, such as vital signs, temperature, perspiration, behaviors, motions or bodily displacements, ability to perform one or more tasks or activities, etc.

Moreover, system 100 may be able to concurrently accommodate a wide variety of electrode configurations 112 at locations 110, such: as different types of electrodes (or sensors), different electrode positions relative to anatomical features of the users (e.g., different positions of the electrodes on the users' heads), different spatial sampling (i.e., different numbers of electrodes or sensors), etc. For example, the measured neurological signals and user feedback from different locations 110 may be collected by server 114 via network 116. (This data collection may be in real time. Alternatively, electronic devices 108 may collect data for a time interval and then may communicate the collected neurological signals to server 114 via network 116.) Then, server 114 may scale (for example, using dynamic time warping), re-sample, remap, normalize, etc. the measured neurological signals to generate modified neurological signals. Therefore, in effect, system 100 may operate in a device-independent manner.

The modified neurological signals can then be analyzed and compared to each other. In particular, server 114 may make the user feedback, the physiological data, the measured neurological signals and/or the modified neurological signals available to different (and, possibly, independent) analysts 118 via network 116. For example, analysts 118 may include: sponsors, researchers, other users, etc. Note that these analysts may analyze the data to identify statistical associations between one or more neurological signals (which are sometimes referred to as ‘neurological signatures,’ ‘physiological events’ or ‘biomarkers’) and the user feedback and/or the physiological data. (Thus, the identification may involve one or more supervised learning techniques, as is known to one of skill in the art.) More generally, system 100 may provide a supervised learning environment in which neurological signatures associated with psychological states and/or physical activities of the users can be determined.

In this way, system 100 may enable users to share data, such as brainwave data, with each other or analysts 118 either in real time, or to aggregate and store such data for later analysis and use.

In an exemplary embodiment, the users may listen to music. Using electrode configurations 112 that are provided by different manufacturers and vendors, the users may monitor their neurological signals while listening to the music. In addition, the users may classify their emotional response to the music by concurrently selecting descriptive adjectives from a finite set of descriptive adjectives (which is the user feedback in this example), for example, using a software program that is installed on and which executes in environments of electronic devices 108. These neurological signals and the descriptive adjectives may be collected and processed by server 114. Then, analysts 118 may analyze the data in an attempt to identify one or more neurological signals that are associated with the users' dynamic temporal cognitive response to the music. For example, analysts 118 may attempt to identify combinations of neurological signals that are statistically associated with the user feedback from users at at least a subset of locations 110. This may allow analysts 118 to determine sets of neurological signals that are associated with the user feedback, such as music that is ‘sultry,’ ‘sexy’ or ‘sad.’ Because music has cultural connotations, note that the collected data may include metadata, such as demographic and/or cultural information about the users, which may be used by analysts 118 when determining the sets of neurological signals.

After being ‘trained’ in this way, system 100 may provide an interactive music-performance platform in which brainwave data from one or more users or listeners is used to influence the score, pitch, or other musical prformance attributes. Ideally, the performer/musical producer would have some idea of the neurological signals (such as electroencephalogram biomarkers) that signify, in an user or in aggregate across a group of users, appreciation or enjoyment of a song. Using this information, the performer/producer may modify the performance in real time so as to maximize the presence of those neurological signals.

In another example, system 100 may be used to implement an interactive card or board game (such as poker), where the players are wearing electrode headsets and each has access to the brainwave data of the other players. The measured neurological signals could be used by a user to discern what type of poker hand a competitor has, i.e., to determine if the competitor is bluffing.

In yet another example, system 100 may be used to synchronize viewing of a political debate (or legal arguments in a court case) in which a large population of voters or potential jurors are wearing electrode headsets and their feedback (such as a positive/negative score) is recorded and aggregated at various points during the argument, thereby giving the debaters feedback on the relative effectiveness of their arguments.

Once the sets of neurological signals have been determined, they can be used to facilitate a variety of services. For example, the neurological signatures may be sold to market research organizations, so that these organizations can obtain neuromarketing data gathered in real time or historically from a large number of different users using different electrode configurations 112 (e.g., different models of brain-sensing devices).

In some embodiments, the neurological signals are measured after neurological stimuli are applied to the users, i.e., the users are first stimulated or driven using electrodes, and subsequently the neurological signals are synchronously or asynchronously measured. Furthermore, after the neurological signatures have been determined during a ‘training’ operating mode of system 100, these neurological signatures may be used open-loop in various applications (such as software programs) that execute on electronic devices 108 to neurologically monitor and/or stimulate the users.

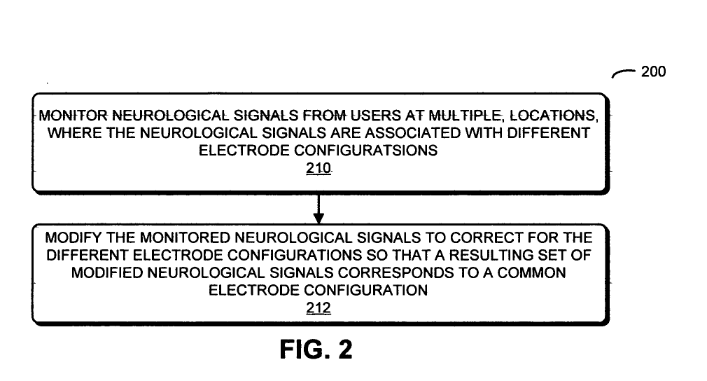

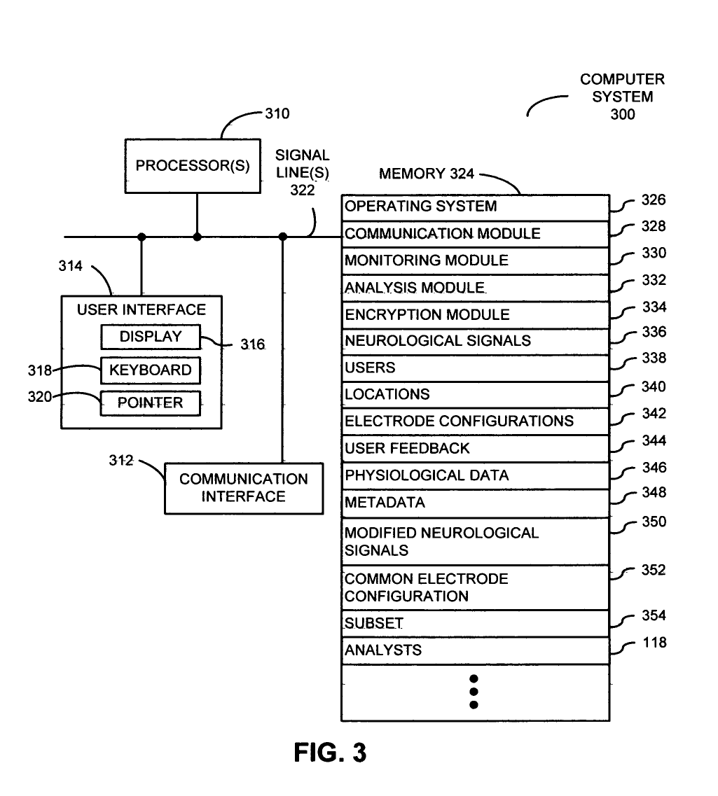

We now describe embodiments of an analysis technique. FIG. 2 presents a flow chart illustrating a method 200 for analyzing neurological signals, which may be performed by a system, such as server 114 in FIG. 1 or computer systems 300 in FIG. 3. During operation, the computer system monitors neurological signals from users at multiple locations, where the neurological signals are associated with different electrode configurations (operation 210). Then, the computer system modifies the monitored neurological signals to correct for the different electrode configurations so that a resulting set of modified neurological signals corresponds to a common electrode configuration (operation 212), thereby facilitating subsequent identification of a subset of the set of modified neurological signals.

Note that the different electrode configurations may include: different types of electrodes, such as different electrode models provided by different manufacturers; different spatial sampling rates; and/or different electrode positions on the users.

In some embodiments of method 200, there may be additional or fewer operations. For example, the set of modified neurological signals may be provided to third parties (such as analysts 118 in FIG. 1) for analysis. Note that these third parties may be other than an operator of server 114 in FIG. 1. Moreover, the order of the operations may be changed, and/or two or more operations may be combined into a single operation.

FIG. 3 presents a block diagram illustrating a computer system 300 (such as server 114 in FIG. 1) that performs method 200 (FIG. 2). Computer system 300 includes one or more processing units or processors 310, a communication interface 312, a user interface 314, and one or more signal lines 322 coupling these components together. Note that the one or more processors 310 may support parallel processing and/or multi-threaded operation, the communication interface 312 may have a persistent communication connection, and the one or more signal lines 322 may constitute a communication bus. Moreover, the user interface 314 may include: a display 316, a keyboard 318, and/or a pointer 320, such as a mouse.

Memory 324 in computer system 300 may include volatile memory and/or non-volatile memory. More specifically, memory 324 may include: ROM, RAM, EPROM, EEPROM, flash memory, one or more smart cards, one or more magnetic disc storage devices, and/or one or more optical storage devices. Memory 324 may store an operating system 326 that includes procedures (or a set of instructions) for handling various basic system services for performing hardware-dependent tasks. Memory 324 may also store procedures (or a set of instructions) in a communication module 328. These communication procedures may be used for communicating with one or more computers and/or servers, including computers and/or servers that may be remotely located with respect to computer system 300.

Memory 324 may also include multiple program modules (or sets of instructions), including: monitoring module 330 (or a set of instructions), analysis module 332 (or a set of instructions) and/or encryption module 334 (or a set of instructions). Note that one or more of these program modules (or sets of instructions) may constitute a computer-program mechanism.

During method 200 (FIG. 2), monitoring module 330 monitors neurological signals 336 from users 338 at locations 340, where neurological signals 336 are associated with different electrode configurations 342. In addition, monitoring module 330 may collect: user feedback 344, physiological data 346, and/or metadata 348.

Then, analysis module 332 modifies neurological signals 336 to correct for different electrode configurations 342 so that a resulting set of modified neurological signals 350 corresponds to a common electrode configuration 352, thereby facilitating subsequent identification of a subset 354 of set of modified neurological signals 350, for example, by analysts 118.

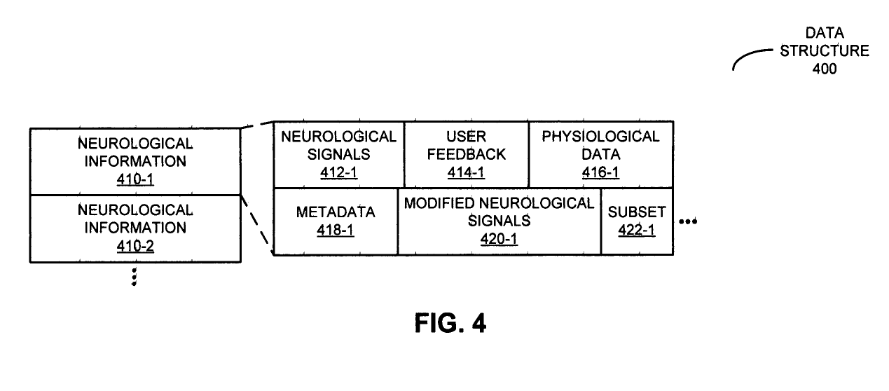

As shown in FIG. 4, which illustrates a data structure 400, the measured and modified neurological signals may be aggregated for use by analysts 118 (FIGS. 1 and 3). For example, neurological information 410-1 may include: neurological signals 412-1, user feedback 414-1, physiological data 416-1, metadata 418-1, modified neurological signals 420-1, and/or subset 422-1 of modified neurological signals 420-1.

Referring back to FIG. 3, instructions in the various modules in memory 324 may be implemented in: a high-level procedural language, an object-oriented programming language, and/or in an assembly or machine language. Note that the programming language may be compiled or interpreted, e.g., configurable or configured, to be executed by the one or more processors 310.

Although computer system 300 is illustrated as having a number of discrete items, FIG. 3 is intended to be a functional description of the various features that may be present in computer system 300 rather than a structural schematic of the embodiments described herein. In practice, and as recognized by those of ordinary skill in the art, the functions of computer system 300 may be distributed over a large number of servers or computers, with various groups of the servers or computers performing particular subsets of the functions. In some embodiments, some or all of the functionality of computer system 300 may be implemented in one or more application-specific integrated circuits (ASICs) and/or one or more digital signal processors (DSPs).

Electronic devices, computers and servers in systems 100 (FIG. 1) and/or computer system 300 may include one of a variety of devices capable of manipulating computer-readable data or communicating such data between two or more computing systems over a network, including: a personal computer, a laptop computer, a tablet computer, a mainframe computer, a portable electronic device (such as a cellular phone or PDA), a server and/or a client computer (in a client-server architecture). Moreover, network 116 (FIG. 1) may include: the Internet, World Wide Web (WWW), an intranet, a cellular-telephone network, LAN, WAN, MAN, or a combination of networks, or other technology enabling communication between computing systems.

System 100 (FIG. 1), computer system 300, and/or data structure 400 may include fewer components or additional components. Moreover, two or more components may be combined into a single component, and/or a position of one or more components may be changed. In some embodiments, the functionality of computer system 300 may be implemented more in hardware and less in software, or less in hardware and more in software, as is known in the art.

The foregoing description is intended to enable any person skilled in the art to make and use the disclosure, and is provided in the context of a particular application and its requirements. Moreover, the foregoing descriptions of embodiments of the present disclosure have been presented for purposes of illustration and description only. They are not intended to be exhaustive or to limit the present disclosure to the forms disclosed. Accordingly, many modifications and variations will be apparent to practitioners skilled in the art, and the general principles defined herein may be applied to other embodiments and applications without departing from the spirit and scope of the present disclosure. Additionally, the discussion of the preceding embodiments is not intended to limit the present disclosure. Thus, the present disclosure is not intended to be limited to the embodiments shown, but is to be accorded the widest scope consistent with the principles and features disclosed herein.

What is claimed is:

1. A computer-implemented method for analyzing neurological signals, comprising:
monitoring neurological signals from users at multiple separate locations, wherein the neurological signals are associated with different electrode configurations; and
using a computer, modifying the monitored neurological signals to correct for the different electrode configurations so that a resulting set of modified neurological signals corresponds to a common electrode configuration so that a subset of the set of modified neurological signals can be identified.

2. The method of claim 1, wherein the different electrode configurations include different types of electrodes.

3. The method of claim 1, wherein the different electrode configurations include different electrode models provided by different manufacturers.

4. The method of claim 1, wherein the different electrode configurations include different spatial sampling rates.

5. The method of claim 1, wherein the different electrode configurations include different electrode positions on the users.

6. The method of claim 1, wherein the neurological signals include electroencephalogram signals.

7. The method of claim 1, wherein the method further comprises analyzing the modified neurological signals based on physiological responses of the users to external stimuli to identify the subset of the set of modified neurological signals.

8. A computer-program product for use in conjunction with a computer system, the computer-program product comprising a non-transitory computer-readable storage medium and a computer-program mechanism embedded therein, to analyze neurological signals, the computer-program mechanism including:
instructions for monitoring neurological signals from users at multiple separate locations, wherein the neurological signals are associated with different electrode configurations; and
instructions for modifying the monitored neurological signals to correct for the different electrode configurations so that a resulting set of modified neurological signals corresponds to a common electrode configuration so that a subset of the set of modified neurological signals can be identified.

9. The computer-program product of claim 8, wherein the different electrode configurations include different types of electrodes.

10. The computer-program product of claim 8, wherein the different electrode configurations include different electrode models provided by different manufacturers.

11. The computer-program product of claim 8, wherein the different electrode configurations include different spatial sampling rates.

12. The computer-program product of claim 8, wherein the different electrode configurations include different electrode positions on the users.

13. The computer-program product of claim 8, wherein the neurological signals include electroencephalogram signals.

14. The computer-program product of claim 8, wherein the computer-program mechanism further includes instructions for analyzing the modified neurological signals based on physiological responses of the users to external stimuli to identify the subset of the set of modified neurological signals.

15. A computer system, comprising:
a processor;
memory; and
a program module, wherein the program module is stored in the memory and configurable to be executed by the processor to analyze neurological signals, the program module including:
instructions for monitoring neurological signals from users at multiple separate locations, wherein the neurological signals are associated with different electrode configurations; and
instructions for modifying the monitored neurological signals to correct for the different electrode configurations so that a resulting set of modified neurological signals corresponds to a common electrode configuration so that a subset of the set of modified neurological signals can be identified.

16. The computer system of claim 15, wherein the different electrode configurations include different types of electrodes.

17. The computer system of claim 15, wherein the different electrode configurations include different electrode models provided by different manufacturers.

18. The computer system of claim 15, wherein the different electrode configurations include different spatial sampling rates.

19. The computer system of claim 15, wherein the different electrode configurations include different electrode positions on the users.

20. The computer system of claim 15, wherein the neurological signals include electroencephalogram signals.
